(19) United States
(12) Patent Application Publication
(10) Pub. No.: US 2012/0065369 A1
(43) Pub. Date: Mar. 15, 2012

(54) LONG LASTING FUSION PEPTIDE INHIBITORS FOR HIV INFECTION

(76) Inventor: OMAR QURAISHI, Westmont (CA)

(21) Appl. No.: 13/047,724

(22) Filed: Mar. 14, 2011

Related U.S. Application Data
(63) Continuation of application No. 11/964,313, filed on Dec. 26, 2007, now abandoned.
(60) Provisional application No. 60/884,865, filed on Jan. 12, 2007.

(51) Int. Cl. C07K 14/76

(52) U.S. Cl. 530/323

(57) ABSTRACT

This invention relates to enfuvirtide peptide derivatives that are inhibitors of viral infection and/or exhibit antifusogenic properties. In particular, this invention relates to enfuvirtide derivatives having inhibiting activity against human immunodeficiency virus (HIV), respiratory syncytial virus (RSV), human parainfluenza virus (HPV), measles virus (MeV), and simian immunodeficiency virus (SIV) with long duration of action for the treatment of the respective viral infections.

This application claims priority to U.S. Provisional Application No. 60/884,865, filed Jan. 12, 2007, the contents of which is incorporated herein in its entirety.

### FIELD OF INVENTION

This invention relates to enfuvirtide peptide derivatives that are inhibitors of viral infection and/or exhibit antifusogenic properties. In particular, this invention relates to enfuvirtide derivatives having inhibiting activity against human immunodeficiency virus (HIV), respiratory syncytial virus (RSV), human parainfluenza virus (HPV), measles virus (MeV), and simian immunodeficiency virus (SIV) with long duration of action for the treatment of the respective viral infections.

### BACKGROUND OF THE INVENTION

Membrane fusion events, while commonplace in normal cell biological processes, are also involved in a variety of disease states, including, for example the entry of enveloped viruses into cells. Peptides are known to inhibit or otherwise disrupt membrane fusion-associated events, including, for example, inhibiting retroviral transmission to uninfected cells.

HIV is a member of the lentivirus family of retroviruses, and there are two prevalent types of HIV, HIV-1 and HIV-2, with various strain of each having been identified. HIV targets CD-4+ cells, and viral entry depends on binding of the HIV protein gp120 to the CD4 glycoprotein and a chemokine receptor on cell surface. Enfuvirtide is a member of the therapeutic class called fusion inhibitors. It is an inhibitor of the structural rearrangement of HIV-1 gp41 that functions by specifically binding to this virus protein extracellularly and thereby blocking the virus from entering the cell. The antiviral activity of enfuvirtide results from its association with a heptad-repeat motif, HR1, within native gp41 on the viral surface.

Enfuvirtide is known to possess antifusogenic activity, i.e., it has the ability to inhibit or reduce the level of membrane fusion events between two or more entities, e.g., virus-cell or cell-cell, relative to the level of membrane fusion that occurs in the absence of the peptide.

### SUMMARY OF THE INVENTION

In accordance with the present invention, there is now provided. Enfuvirtide peptide derivatives having an extended in vivo half-life when compared with the corresponding unmodified enfuvirtide peptide sequence. The present invention includes compounds that include an amino acid sequence of enfuvirtide conjugated, e.g., with a linker illustrated below, which are capable of reacting with thiol groups on a blood component, either in vivo or ex vivo, to form a stable covalent bond. Preferred blood components comprise proteins such as immunoglobulins, including IgG and IgM, serum albumin, ferritin, steroid binding proteins, transferrin, thyroxin binding protein, α-2-macroglobulin etc., serum albumin and IgG being more preferred, and serum albumin, e.g., human serum albumin being the most preferred.

In another aspect of the invention, there is provided a pharmaceutical composition comprising the enfuvirtide peptide derivatives, e.g., the enfuvirtide peptide derivatives described herein in combination with a pharmaceutically acceptable carrier. Such composition is useful for inhibiting the activity of HIV, RSV, HPV, MeV or SIV.

In a further embodiment of the present invention, there is provided a method for inhibiting the activity of HIV, RSV, HPV, MeV or SIV. The method comprises administering to a subject, preferably a mammal, an effective amount of the enfuvirtide peptide derivatives, e.g., the enfuvirtide peptide derivatives described herein, alone or in combination with a pharmaceutical carrier.

In a further aspect of the present invention, there is provided a conjugate comprising a enfuvirtide peptide derivative covalently bonded to a blood component through a compound having a structure of Formulae I-V.

In a further aspect of the present invention, there is provided a method for extending the in vivo half-life of an enfuvirtide peptide in a subject, the method comprising covalently bonding, e.g., through a compound of Formulae I-V to a blood component. In one embodiment, the enfuvirtide peptide is covalently bound to a blood component through a compound provided below:

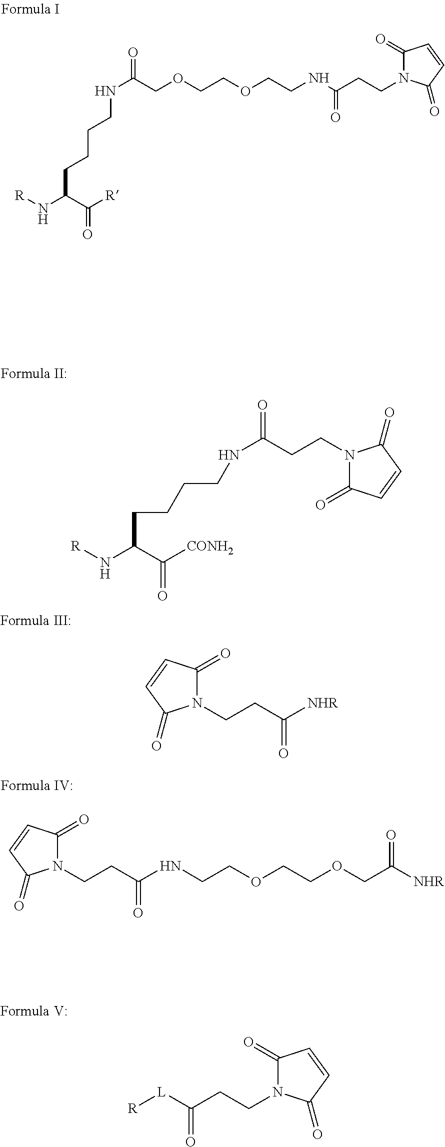

### DETAILED DESCRIPTION OF THE INVENTION

The present invention meets these and other needs and is directed to enfuvitride peptides derivatives having anti-viral activity and/or anti-fusogenic activity. These enfuvitride peptides derivatives provide for an increased stability in vivo and a reduced susceptibility to peptidase or protease degradation. Preferably, the enfuvitride peptide derivatives are conjugated with a blood component, e.g., serum albumin, e.g., human serum albumin, by covalent bond through a compound. In some embodiments, the blood component is bound to the enfuvitride by a compound of Formulae I-V. Enfuvitride peptide derivatives, e.g., described herein, minimize the need for more frequent, or even continual, administration of the peptides. The present enfuvitride peptide derivatives can be used, e.g., as a prophylactic against and/or treatment for infection of a number of viruses, including human immunodeficiency virus (HIV), human respiratory syncytial virus (RSV), human parainfluenza virus (HPV), measles virus (MeV) and simian immunodeficiency virus (SIV).

The modification made to the native enfuvitride peptide sequence allows it to react with available thiol groups on blood components to form stable covalent bonds. In one embodiment of the invention, the blood component comprises a blood protein, including a mobile blood protein such as albumin, which is most preferred.

The enfuvitride peptide derivatives inhibit viral infection of cells, by, for example, inhibiting cell-cell fusion or free virus infection. The route of infection may involve membrane fusion, as occurs in the case of enveloped or encapsulated viruses, or some other fusion event involving viral and cellular structures.

The blood components to which the present anti-viral enfuvitride peptide derivatives covalently bond may be either fixed or mobile. Fixed blood components are non-mobile blood components and include tissues, membrane receptors, interstitial proteins, fibrin proteins, collagens, platelets, endothelial cells, epithelial cells and their associated membrane and membraneous receptors, somatic body cells, skeletal and smooth muscle cells, neuronal components, osteocytes and osteoclasts and all body tissues especially those associated with the circulatory and lymphatic systems. Mobile blood components are blood components that do not have a fixed situs for any extended period of time, generally not exceeding 5 minutes, and more usually one minute. These blood components are not membrane-associated and are present in the blood for extended periods of time in a minimum concentration of at least 0.1 μg/ml. Mobile blood components include serum albumin, transferrin, ferritin and immunoglobulins such as IgM and IgG. The half-life of mobile blood components is at least about 12 hours.

Protective groups may be required during the synthesis process of the enfuvitride peptide derivative. These protective groups are conventional in the field of peptide synthesis, and can be generally described as chemical moieties capable of protecting the peptide derivative from reacting with other functional groups. Various protective groups are available commercially, and examples thereof can be found in U.S. Pat. No. 5,493,007 which is hereby incorporated by reference. Typical examples of suitable protective groups include acetyl, fluorenylmethyloxycarbonyl (FMOC), t-butyloxycarbonyl (BOC), benzyloxy-carbonyl (CBZ), etc. In addition, Table 1 provides both the three letter and one letter abbreviations for amino acids.

TABLE 1
NATURAL AMINO ACIDS AND THEIR ABBREVIATIONS

| | 3-letter | 1-letter |
|---|---|---|
| Name | abbreviation | abbreviation |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The enfuvitirde peptide derivatives may be administered in vivo such that conjugation with blood components occurs in vivo, or they may be first conjugated to blood components in vitro and the resulting conjugated derivative administered in vivo.

The present invention takes advantage of the properties of existing anti-viral and antifusogenic peptides. The viruses that may be inhibited by the peptides include, but are not limited to all strains of viruses listed, e.g., in U.S. Pat. No. 6,013,263 and U.S. Pat. No. 6,017,536 at Tables V-VII and IX-XIV therein. These viruses include, e.g., human retroviruses, including HIV-1, HIV-2, and human T-lympocyte viruses (HTLV-I and HTLV-II), and non-human retroviruses, including bovine leukosis virus, feline sarcoma virus, feline leukemia virus, simian immunodeficiency virus (SIV), simian sarcoma virus, simian leukemia, and sheep progress pneumonia virus. Non-retroviral viruses may also be inhibited by the enfuvitride peptide derivatives, including human respiratory syncytial virus (RSV), canine distemper virus, Newcastle Disease virus, human parainfluenza virus (HPIV), influenza viruses, measles viruses (MeV), Epstein-Barr viruses, hepatitis B viruses, and simian Mason-Pfizer viruses. Non-enveloped viruses may also be inhibited by the enfuvitride peptide derivatives, and include, but are not limited to, picornaviruses such as polio viruses, hepatitis A virus, enteroviruses, echoviruses, coxsackie viruses, papovaviruses such as papilloma virus, parvoviruses, adenoviruses, and reoviruses.

Described are modified enfuvitride peptide sequences to confer to this peptide improved bio-availability, extended half-life and better distribution through selective conjugation of the peptide onto a protein carrier without substantially modifying the peptide's anti-viral properties. A preferred carrier would be albumin conjugated through its free thiol.

The enfuvitride peptide preferably has the following amino acid sequence:

Ac-Tyr-Thr-Ser-Leu-Ile-His-Ser-Leu-Ile-Glu-Glu-Ser-Gln-Asn-Gln-Gln-
Glu-Lys-Asn-Glu-Gln-Glu-Leu-Leu-Glu-Leu-Asp-Lys-Trp-Ala-Ser-Leu-Trp-
Asn-Trp-Phe-NH₂

Known modifications the enfuvitride peptide are also contemplated herein.

The enfuvitride peptide derivatives described herein can be designed to specifically react with thiol groups on mobile blood proteins. Such reaction is established by covalent bonding of the peptide modified with a maleimide link to a thiol group on a mobile blood protein such as serum albumin or IgG.

Thiol groups being less abundant in vivo than, for example, amino groups, the maleimide-modified enfuvitride peptide, e.g., as described herein, will covalently bond to fewer proteins. For example, in albumin (the most abundant blood protein) there is only a single thiol group. Thus, a enfuvitirde-maleimide-albumin conjugate will tend to comprise approximately a 1:1 molar ratio of enfuvitride peptide to albumin. In addition to albumin, IgG molecules (class II) also have free thiols. Since IgG molecules and serum albumin make up the majority of the soluble protein in blood they also make up the majority of the free thiol groups in blood that are available to covalently bond to the enfuvitride peptide derivative.

Further, even among free thiol-containing blood proteins, including IgGs, specific labeling with a maleimide leads to the preferential formation of a enfuvitride-maleimide-albumin conjugate due to the unique characteristics of albumin itself. The single free thiol group of albumin, highly conserved among species, is located at amino acid residue 34 (Cys³⁴). It has been demonstrated recently that the Cys³⁴ of albumin has increased reactivity relative to free thiols on other free thiol-containing proteins. This is due in part to the very low pK value of 5.5 for the Cys³⁴ of albumin. This is much lower than typical pK values for cysteine residues in general, which are typically about 8. Due to this low pK, under normal physiological conditions Cys³⁴ of albumin is predominantly in the ionized form, which dramatically increases its reactivity. In addition to the low pK value of Cys³⁴, another factor which enhances the reactivity of Cys³⁴ is its location, which is in a hydrophobic pocket close to the surface of one loop of region V of albumin. This location makes Cys³⁴ very available to ligands of all kinds, and is an important factor in Cys³⁴'s biological role as free radical trap and free thiol scavenger. These properties make Cys³⁴ highly reactive with maleimide-enfuvitride, and the reaction rate acceleration can be as much as 1000-fold relative to rates of reaction of maleimide-enfuvitride with other free-thiol containing proteins.

Another advantage of enfuvitride-maleimide-albumin conjugates is the reproducibility associated with the 1:1 loading of enfuvitride to albumin specifically at Cys³⁴. Other techniques, such as glutaraldehyde, DCC, EDC and other chemical activations of, e.g, free amines, lack this selectivity. For example, albumin contains 52 lysine residues, 25-30 of which are located on the surface of albumin and therefore accessible for conjugation. Activating these lysine residues, or alternatively modifying enfuvitride to couple through these lysine residues, results in a heterogenous population of conjugates. Even if 1:1 molar ratios of enfuvitride to albumin are employed, the yield will consist of multiple conjugation products, some containing 0, 1, 2 or more enfuvitride per albumin, and each having enfuvitride randomly coupled at any one or more of the 25-30 available lysine sites. Given the numerous possible combinations, characterization of the exact composition and nature of each conjugate batch becomes difficult, and batch-to-batch reproducibility is all but impossible, making such conjugates less desirable as a therapeutic. Additionally, while it would seem that conjugation through lysine residues of albumin would at least have the advantage of delivering more therapeutic agent per albumin molecule, studies have shown that a 1:1 ratio of therapeutic agent to albumin is preferred. See, e.g., Stehle, et al., “The Loading Rate Determines Tumor Targeting properties of Methotrexate-Albumin Conjugates in Rats,” Anti-Cancer Drugs, Vol. 8, pp. 677-685 (1988), incorporated herein in its entirety.

Through controlled administration of enfuvitride peptide derivatives, such as those described herein, in vivo, one can control the specific labeling of albumin and IgG in vivo. In typical administrations, 80-90% of the administered enfuvitride derivatives will label albumin and less than 5% will label IgG. Trace labeling of free thiols such as glutathione will also occur. Such specific labeling is preferred for in vivo use as it permits an accurate calculation of the estimated half-life of enfuvitride.

The enfuvitride peptide can be conjuagted to a blood component by a lyseine residue that occurs in enfuvitride or can be conjuagted at a site added, e.g., through peptide synthesis.

In addition to providing controlled specific in vivo labeling, the enfuvitride peptide derivatives can provide specific labeling of serum albumin and IgG ex vivo. Such ex vivo labeling involves the addition of the enfuvitride derivatives to blood, serum or saline solution containing serum albumin and/or IgG. Once conjugation has occurred ex vivo with the enfuvitride derivative, the blood, serum or saline solution can be readministered to the patient's blood for in vivo treatment, or lyophilized.

An enfuvitride derivative can be synthesized by standard methods of solid phase peptide chemistry well known to any one of ordinary skill in the art. For example, the peptide may be synthesized by solid phase chemistry techniques following the procedures described by Steward et al. in Solid Phase Peptide Synthesis, 2nd Ed., Pierce Chemical Company, Rockford, Ill., (1984) using a Rainin PTI Symphony synthesizer. Similarly, peptides fragments may be synthesized and subsequently combined or linked together to form the enfuvitride peptide sequence (segment condensation).

For solid phase peptide synthesis, a summary of the many techniques may be found in Stewart et al. in “Solid Phase Peptide Synthesis”, W. H. Freeman Co. (San Francisco), 1963 and Meienhofer, Hormonal Proteins and Peptides, 1973, 2 46. For classical solution synthesis, see for example Schroder et al. in “The Peptides”, volume 1, Acacemic Press (New York). In general, such method comprise the sequential addition of one or more amino acids or suitably protected amino acids to a growing peptide chain on a polymer. Normally, either the amino or carboxyl group of the first amino acid is protected by a suitable protecting group. The protected and/or derivatized amino acid is then either attached to an inert solid support or utilized in solution by adding the next amino acid in the sequence having the complimentary (amino or carboxyl) group suitably protected and under conditions suitable for forming the amide linkage. The protecting group is then removed from this newly added amino acid residue and the next amino acid (suitably protected) is added, and so forth.

After all the desired amino acids have been linked in the proper sequence, any remaining protecting groups (and any solid support) are cleaved sequentially or concurrently to afford the final peptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide.

The enfuvitride peptide derivatives can be synthesized by solid phase peptide synthesis wherein the amino acid α-N-terminal is protected by an acid or base sensitive group. Such protecting groups should have the properties of being stable to the conditions of peptide linkage formation while being readily removable without destruction of the growing peptide chain or racemization of any of the chiral centers contained therein. Examples of N-protecting groups and carboxy-protecting groups are disclosed in Greene, “Protective Groups In Organic Synthesis,” (John Wiley & Sons, New York pp. 152-186 (1981)), which is hereby incorporated by reference. Examples of N-protecting groups comprise, without limitation, loweralkanoyl groups such as formyl, acetyl (“Ac”), propionyl, pivaloyl, t-butylacetyl and the like; other acyl groups include 2-chloroacetyl, 2-bromoacetyl, trifluoroacetyl, trichloroacetyl, phthalyl, o-nitrophenoxy-acetyl, -chlorobutyryl, benzoyl, 4-chlorobenzoyl, 4-bromobenzoyl, 4-nitrobenzoyl and the like; sulfonyl groups such as benzenesulfonyl, p-toluenesulfonyl, o-nitrophenylsulfonyl, 2,2,5,7,8-pentamethylchroman-6-sulfonyl (pmc), and the like; carbamate forming groups such as t-amyloxycarbonyl, benzyloxycarbonyl, p-chlorobenzyloxycarbonyl, p-methoxy-benzyloxycarbonyl, p-nitrobenzyloxycarbonyl, 2-nitrobenzyloxycarbonyl, p-bromo-benzyloxycarbonyl, 3,4-dimethoxybenzyloxycarbonyl, 3,5-dimethoxybenzyloxycarbonyl, 2,4-dimethoxybenzyloxycarbonyl, 4-ethoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (boc), diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, isobornyloxycarbonyl, cyclopentyloxycarbonyl, adamantyloxycarbonyl, cyclohexyloxycarbonyl, phenylthiocarbonyl and the like; arylalkyl groups such as benzyl, biphenylisopropyloxycarbonyl, triphenylmethyl, benzyloxymethyl, 9-fluorenylmethyloxycarbonyl (Fmoc) and the like and silyl groups such as trimethylsilyl and the like. Preferred α-N-protecting group are o-nitrophenylsulfenyl; 9-fluorenylmethyl oxycarbonyl; t-butyloxycarbonyl (boc), isobornyloxycarbonyl; 3,5-dimethoxybenzyloxycarbonyl; t-amyloxycarbonyl; 2-cyano-t-butyloxycarbonyl, and the like, 9-fluorenyl-methyloxycarbonyl (Fmoc) being more preferred, while preferred side chain N-protecting groups comprise 2,2,5,7,8-pentamethylchroman-6-sulfonyl (pmc), nitro, p-toluenesulfonyl, 4-methoxybenzene-sulfonyl, Cbz, Boc, and adamantyloxycarbonyl for side chain amino groups like lysine and arginine; benzyl, o-bromobenzyloxycarbonyl, 2,6-dichlorobenzyl, isopropyl, t-butyl (t-Bu), cyclohexyl, cyclopenyl and acetyl (Ac) for tyrosine; t-butyl, benzyl and tetrahydropyranyl for serine; trityl, benzyl, Cbz, p-toluenesulfonyl and 2,4-dinitrophenyl for histidine; formyl for tryptophan; benzyl and t-butyl for aspartic acid and glutamic acid; and triphenylmethyl (trityl) for cysteine.

A carboxy-protecting group conventionally refers to a carboxylic acid protecting ester or amide group. Such carboxy protecting groups are well known to those skilled in the art, having been extensively used in the protection of carboxyl groups in the penicillin and cephalosporin fields as described in U.S. Pat. No. 3,840,556 and U.S. Pat. No. 3,719,667, the disclosures of which are hereby incorporated herein by reference. Representative carboxy protecting groups comprise, without limitation, C₁-C₈ loweralkyl; arylalkyl such as phenethyl or benzyl and substituted derivatives thereof such as alkoxybenzyl or nitrobenzyl groups; arylalkenyl such as phenylethenyl; aryl and substituted derivatives thereof such as 5-indanyl; dialkylaminoalkyl such as dimethylaminoethyl; alkanoyloxyalkyl groups such as acetoxymethyl, butyryloxymethyl, valeryloxymethyl, isobutyryloxymethyl, isovaleryloxymethyl, 1-(propionyloxy)-1-ethyl, 1-(pivaloyloxyl)-1-ethyl, 1-methyl-1-(propionyloxy)-1-ethyl, pivaloyloxymethyl, propionyloxymethyl; cycloalkanoyloxyalkyl groups such as cyclopropylcarbonyloxymethyl, cyclobutylcarbonyloxymethyl, cyclopentylcarbonyloxymethyl, cyclohexylcarbonyloxy-methyl; aroyloxyalkyl such as benzoyloxymethyl, benzoyloxyethyl; arylalkylcarbonyloxyalkyl such as benzylcarbonyloxymethyl, 2-benzylcarbonyloxyethyl; alkoxycarbonylalkyl or cycloalkyloxycarbonyl-alkyl such as methoxycarbonylmethyl, cyclohexyloxycarbonylmethyl, 1-methoxy-carbonyl-1-ethyl; alkoxycarbonyloxyalkyl or cycloalkyloxycarbonyloxyalkyl such as methoxycarbonyloxymethyl, t-butyloxycarbonyl-oxymethyl, 1-ethoxycarbonyloxy-1-ethyl, 1-cyclohexyloxycarbonyloxy-1-ethyl; aryloxy-carbonyloxyalkyl such as 2-(phenoxycarbonyloxy)ethyl, 2-(5-indanyloxycarbonyloxy)-ethyl; alkoxyalkylcarbonyl-oxyalkyl such as 2-(1-methoxy-2-methylpropan-2-oyloxy)-ethyl; arylalkyloxycarbonyl-oxyalkyl such as 2-(benzyloxycarbonyloxy)ethyl; arylalkenyloxycarbonyloxyalkyl such as 2-(3-phenylpropen-2-yloxycarbonyloxy)ethyl; alkoxycarbonylaminoalkyl such as t-butyloxycarbonylaminomethyl; alkylaminocarbonyl-aminoalkyl such as methylamino-carbonylaminomethyl; alkanoylaminoalkyl such as acetylaminomethyl; heterocyclic-carbonyloxyalkyl such as 4-methylpiperazinyl-carbonyloxymethyl; dialkylamino-carbonylalkyl such as dimethylaminocarbonylmethyl, diethylaminocarbonylmethyl; (5-(loweralkyl)-2-oxo-1,3-dioxolen-4-yl)alkyl such as (5-t-butyl-2-oxo-1,3-dioxolen-4-yl)methyl; and (5-phenyl-2-oxo-1,3-dioxolen-4-yl)alkyl such as (5-phenyl-2-oxo-1,3-dioxolen-4-yl)methyl. Representative amide carboxy protecting groups comprise, without limitation, aminocarbonyl and loweralkylaminocarbonyl groups. Of the above carboxy-protecting groups, loweralkyl, cycloalkyl or arylalkyl ester, for example, methyl ester, ethyl ester, propyl ester, isopropyl ester, butyl ester, sec-butyl ester, isobutyl ester, amyl ester, isoamyl ester, octyl ester, cyclohexyl ester, phenylethyl ester and the like or an alkanoyloxyalkyl, cycloalkanoyloxyalkyl, aroyloxyalkyl or an arylalkylcarbonyloxyalkyl ester are preferred. Preferred amide carboxy protecting groups are loweralkylamino-carbonyl groups.

In the solid phase peptide synthesis method, the α-C-terminal amino acid is attached to a suitable solid support or resin. Suitable solid supports useful for the above synthesis are those materials that are inert to the reagents and reaction conditions of the stepwise condensation-deprotection reactions, as well as being insoluble in the media used. The preferred solid support for synthesis of α-C-terminal carboxy peptides is 4-hydroxymethylphenoxyacetyl-4′-methylbenzyhydrylamine resin (HMP resin). The preferred solid support for α-C-terminal amide peptides is an Fmoc-protected Ramage resin, manufactured and sold by Bachem Inc., California.

At the end of the solid phase synthesis, the peptide is removed from the resin and deprotected, either in successive operations or in a single operation. Removal of the peptide and deprotection can be accomplished conventionally in a single operation by treating the resin-bound polypeptide with a cleavage reagent comprising thioanisole, triisopropyl silane, phenol, and trifluoroacetic acid. In cases wherein the α-C-terminal of the peptide is an alkylamide, the resin is cleaved by aminolysis with an alkylamine. Alternatively, the peptide may be removed by transesterification, e.g. with methanol, followed by aminolysis or by direct transamidation. The protected peptide may be purified at this point or taken to the next step directly. The removal of the side chain protecting groups is accomplished using the cleavage mixture described above. The fully deprotected peptide can be purified by a sequence of chromatographic steps employing any or all of the following types: ion exchange on a weakly basic resin (acetate form); hydrophobic adsorption chromatography on underivatized polystyrene-divinylbenzene (such as Amberlite XAD™); silica gel adsorption chromatography; ion exchange chromatography on carboxymethylcellulose; partition chromatography, e.g. on Sephadex G25™, LH20™ or countercurrent distribution; high performance liquid chromatography (HPLC), especially reverse-phase HPLC on octyl- or phenyl/hexylsilyl-silica bonded phase column packing. Anyone of ordinary skill in the art will be able to determine easily what would be the preferred chromatographic steps or sequences required to obtain acceptable purification of the enfuvitride peptide.

Molecular weights of these peptides are determined using Electrospray mass spectroscopy.

The enfuvitride peptide derivatives may be used alone or in combination to optimize their therapeutic effects. They can be administered in a physiologically acceptable medium, e.g. deionized water, phosphate buffered saline (PBS), saline, aqueous ethanol or other alcohol, plasma, proteinaceous solutions, mannitol, aqueous glucose, alcohol, vegetable oil, or the like. Other additives which may be included include buffers, where the media are generally buffered at a pH in the range of about 5 to 10, where the buffer will generally range in concentration from about 50 to 250 mM, salt, where the concentration of salt will generally range from about 5 to 500 mM, physiologically acceptable stabilizers, and the like. The compositions may be lyophilized for convenient storage and transport.

The enfuvitride peptide derivatives may be administered parenterally, such as intravascularly (IV), intraarterially (IA), intramuscularly (IM), subcutaneously (SC), or the like. Administration may in appropriate situations be by transfusion. In some instances, where reaction of the functional group is relatively slow, administration may be oral, nasal, rectal, transdermal or aerosol, where the nature of the conjugate allows for transfer to the vascular system. Usually a single injection will be employed although more than one injection may be used, if desired. The peptide derivative may be administered by any convenient means, including syringe, trocar, catheter, or the like. The particular manner of administration will vary depending upon the amount to be administered, whether a single bolus or continuous administration, or the like. Preferably, the administration will be intravascularly, where the site of introduction is not critical to this invention, preferably at a site where there is rapid blood flow, e.g., intravenously, peripheral or central vein. Other routes may find use where the administration is coupled with slow release techniques or a protective matrix. The enfuvitride peptide derivative is preferably effectively distributed in the blood, so as to be able to react with the blood components. The concentration of the conjugate will vary widely, generally ranging from about 1 pg/ml to 50 mg/ml. The total administered intravascularly will generally be in the range of about 0.1 mg/ml to about 10 mg/ml, more usually about 1 mg/ml to about 5 mg/ml.

By bonding to long-lived components of the blood, such as immunoglobulin, serum albumin, red blood cells and platelets, a number of advantages ensue. The activity of the enfuvitride peptide derivatives can be extended for days to weeks. Only one administration need to be given during this period of time. Greater specificity can be achieved, since the active compound will be primarily bound to large molecules, where it is less likely to be taken up intracellularly to interfere with other physiological processes.

The formation of the covalent bond between the blood component may occur in vivo or ex vivo. For ex vivo covalent bond formation, the enfuvitride peptide derivative is added to blood serum or a saline solution containing purified blood components such as human serum albumin or IgG, to permit covalent bond formation between the derivative and the blood component. Preferably, the enfuvitride peptide derivative is reacted with human serum albumin in saline solution. After formation of the conjugate, the latter may be administered to the subject or lyophilized.

The blood of the mammalian host may be monitored for the activity of the enfuvitride peptide and/or presence of the enfuvitride peptide derivatives. By taking a blood sample from the host at different times, one may determine whether enfuvamide peptide derivative has become bonded to the long-lived blood components in sufficient amount to be therapeutically active and, thereafter, the level of enfuvitride derivative in the blood. If desired, one may also determine to which of the blood components enfuvitride is covalently bonded. Monitoring may also take place by using assays of enfuvitride activity, HPLC-MS or antibodies directed to enfuvitride.

The following examples are provided to illustrate preferred embodiments of the invention and shall by no means be construed as limiting its scope.

The enfuvatride peptide derivatives can be administered to patients according to the methods described below and other methods known in the art. Effective therapeutic dosages of the enfuvitride peptide derivatives can be determined through procedures well known by those in the art and will take into consideration any concerns over potential toxicity of enfuvitride.

The enfuvitride peptide derivative can also be administered prophylactically to previously uninfected individuals. This can be advantageous in cases where an individual has been subjected to a high risk of exposure to a virus, as can occur when individual has been in contact with an infected individual where there is a high risk of viral transmission. This can be especially advantageous where there is known cure for the virus, such as the HIV virus. As an example, prophylactic administration of an enfuvitride peptide derivative would be advantageous in a situation where a health care worker has been exposed to blood from an HIV-infected individual, or in other situations where an individual engaged in high-risk activities that potentially expose that individual to the HIV virus.

The invention having been fully described can be further appreciated and understood with reference to the following non-limiting examples.

### EXAMPLES

CjC-1, an albumin-conjugated peptide fusion inhibitor was modeled on the fusion inhibitor T-20 (enfuvirtide). It was designed to facilitate less frequent dosing in humans by increasing in vivo half-life (>10 days) and sustaining plasma levels compared with unconjugated peptide. The drug is a 1:1 covalent conjugate with specific attachment of the fusion-inhibiting peptide on cysteine 34 of albumin. The peptide is predicted to bind to a different region of the helical groove of the gp41 N-terminal heptad repeat than does T-20.

The activity of a single preexposure dose of CjC-1 was evaluated in SCID-hu Thy/Liv mice infected with HIV-1 NL4-3 24 h after subcutaneous administration of the conjugate. Thy/Liv implants were inoculated with 1,000 TCID50 of HIV-1 by direct injection and were collected 3 weeks later. The implants were dispersed into single-cell suspensions and assessed for p24 by ELISA, for HIV-1 RNA by the branched DNA assay, and for depletion of thymocyte subsets by multiparameter flow cytometry.

Treatment with one dose of 200 mg/kg CjC-1 (fusion inhibitor concentration) reduced implant viral RNA by 3.3 log¹⁰ and p24 by >95% compared to untreated infected mice. Four of six treated mice had no detectable p24 and one of six mice had no detectable p24 or HIV-1 RNA. A single dose of CjC-1 in these mice also protected immature and mature T-cells from virus-mediated cytopathicity and depletion and reduction in the CD4/CD8 ratio. In stark contrast to our findings with the CjC-1, a single preexposure dose of Truvada (200 mg/kg tenofovir DF and 133 mg/kg emtricitabine) 24 h before virus inoculation reduced viral RNA by only 0.8 log¹⁰ and p24 by 59% and was substantially less effective in preventing thymocyte depletion. It was observed that similarly potent activity of CjC-1 in SCID-hu mice treated once daily with 20 mg/kg beginning 3 days after virus inoculation. In other SCID-hu studies, CjC-1 was 30-100 times more potent that T-20 when given twice-daily beginning 1 day before inoculation.

This confirms the potent in vivo anti-HIV activity of CjC-1. The potent activity observed for a single preexposure dose supports further preclinical and clinical development of this long-acting fusion inhibitor and confirms the usefulness of the SCID-hu Thy/Liv model for evaluation of in vivo antiretroviral efficacy.

What is claimed:

1. An enfuvitride peptide derivative conjugated to albumin through a reactive group coupled to the enfuvitride peptide and covalently bound to albumin.

2. The conjugate of claim 1, wherein the reactive group is a maleimido-containing group.

3. The conjugate of claim 2, wherein the reactive group is 3-Maleimidopropionic acid (MPA).

4. The conjugate of claim 2, wherein the reactive group is coupled to an amino acid of the enfuvitride eptide via a linker.

5. The conjugate of claim 4, wherein said linker that reacts with the reactive group and the peptide selected from the group consisting of ethylenediamine (EDA), AEES-AEES, 2-[2-(2-amino)ethoxy)]ethoxy acetic acid (AEEA), AEEA-AEEA and NH₂—(CH₂)ₙ—COOH where n is an integer between 1 and 20.
